(19) United States
(12) Patent Application Publication
(10) Pub. No.: US 2024/0156664 A1
(43) Pub. Date: May 16, 2024

(54) EXOSKIN

(71) Applicant: MOTORSKINS UG, Berlin (DE)

(72) Inventors: Facundo GUTIERREZ, Berlin (DE); Juan OPITZ-SILVA, Berlin (DE); José VILLATORO, Berlin (DE); Nils JANSSEN, Berlin (DE)

(21) Appl. No.: 18/281,287

(22) PCT Filed: Apr. 14, 2022
(86) PCT No.: PCT/EP2022/060103

§ 371 Date: Sep. 9, 2023

(30) Foreign Application Priority Data
Apr. 15, 2021 (DE) 10 2021 109 526.3

(51) Int. Cl. A61H 3/00; A61F 13/08; B25J 9/00; B25J 9/14

(52) U.S. Cl. CPC A61H 3/00; A61F 13/08; B25J 9/0006; B25J 9/142; A61H 2201/1238; A61H 2201/1642; A61H 2201/165; A61H 2201/5051

(57) ABSTRACT

A wearable exoskin (100), which generates energy by an external force of a user applied to the exoskin (100) and utilizes that a energy to support the movement of the user, comprises at least one fluid circuit (30), at least one interface (10) which is fluidly connected to the at least one fluid circuit (30) and at least one actuator (20) which is fluidly connected to the at least one fluid circuit (30). The at least one fluid circuit (30) comprises a displaceable fluid (40). The fluid actuator (20) is changeable in volume and switches from a first state (with a first volume) to a second state (with a second volume), which is different from the first state, in reaction to the flow of the displaceable fluid (40). The displacement of the fluid (40) is caused by an interaction of the user with the at least one interface (10), such as the application of an external force. The exoskin (100) can be used in a compression garment, such as used on a lower limb.

### CROSS REFERENCE TO RELATED APPLICATIONS

This application claims priority of German application DE 10 2021 109 526.3 filed on 15 Apr. 2021. The entire disclosure of the German application DE 10 2021 109 526.3 is hereby incorporated herein by reference.

### FIELD OF THE INVENTION

The field of the invention concerns soft robotics technology and more particularly a wearable exoskin for supporting movement of body parts of a user and reduce fatigue of muscles.

### PRIOR ART

A textile actuator worn by a user is known from the European patent application No. EP 3 631 212 A1. The textile actuator comprises a textile envelope that defines a chamber made fluid-impermeable by a fluid-impermeable bladder contained in the textile envelope and/or a fluid-impermeable structure incorporated into the textile envelope.

The US patent application No. US 2014/318118 A1 discloses a flexible robotic actuator comprising a flexible, non-extensible material in the form of a sheet or thin film, a flexible inflatable layer in the form of a thin film or sheet in facing relationship with the strain-limiting layer. The flexible inflatable layer is selectively adhered to the strain-limiting layer, and a portion of an un-adhered region between the strain-limiting layer and the inflatable layer defines a pressurizable channel. At least one fluid inlet is in fluid communication with the pressurizable channel.

The US patent application US 2017/202724 A1 discloses assistive flexible suits, flexible suit systems, and methods for making and control thereof to assist human mobility.

The method involves monitoring an output of a sensor when a person moves in a first controlled movement environment. A predefined gait event is identified using the output of the sensor. An actuation profile of an actuator is adjusted. Acts of monitoring, identifying, and adjusting are continuously performed until the actuation profile of the actuator generates beneficial moment about a joint to promote an improvement in gait. A controller is set to implement the actuation profile of the actuator.

German patent application DE 10 2016 118 999 A1 discloses an actuator-damper unit for use in orthotic or prosthetic devices. The actuator-damper unit includes a housing which may be fastened onto on the orthotic or prosthetic device. A cylinder of fixed volume is formed in the housing and includes a piston which divides the cylinder into two fluid chambers located on each side of the piston. Fluid lines connect the two fluid chambers to each other. Control valves are disposed on the fluid lines and the control valves are connected to a control device. The control device can be operated manually and allows the opening or closure of the control valves. By the opening or closure of the control valves, the piston moves laterally in the cylinder from a first position to a second position. The movement of the piston from a first position to a second position induces a variation of the volume of the fluid chambers in the housing, resulting in the entry or the exit of the fluid into or out of the fluid chambers.

The prior art does not teach a system wherein the energy to power a wearable movement-supporting device is generated by the user when wearing the device.

### BRIEF DESCRIPTION OF THE INVENTION

The present document describes a wearable exoskin which generates energy by an external force of a user applied to the exoskin and utilizes that energy to support the movement of the user.

In one aspect, the exoskin comprises at least one fluid circuit, at least one interface which is fluidly connected to the at least one fluid circuit and at least one fluid actuator which is fluidly connected to the at least one fluid circuit. The at least one fluid circuit comprises a displaceable fluid. The at least one fluid actuator is changeable in volume and switches from a first state with a first volume to a second state with a second volume, which is different from the first state, in reaction to the flow of the displaceable fluid. The displacement of the fluid is caused by an interaction of the user with the at least one interface, such as the application of an external force.

If the user interacts with the at least one interface, for example by stepping with the heel or ball of the foot on the interface, the fluid in the interface will be propelled outward of the interface toward the fluid circuit and thus increasing fluid pressure in the circuit. The fluid actuator is changeable in volume and can absorb the fluid. Unlike in the prior art, there are no mechanical parts, such as pistons, in the exoskin which are liable to mechanical failure.

In one aspect, the exoskin further comprises at least one valve located in the at least one fluid circuit for cutting off the flow of the fluid in the at least one fluid circuit. The at least one valve thereby forces the displaceable fluid to move in one specific direction.

In one aspect, the exoskin further comprises a fluid bladder located in the at least one fluid circuit to compensate the increase of fluid pressure. If the valve is in a closed state and the pressure increases, the fluid bladder stretches and compensates the pressure. Pressure energy can thus be stored in the system as well as avoiding a bursting of the system due to overpressure.

In one aspect, the exoskin is made of a textile material. The textile material is at least partially coated and/or laminated with a fluid-impermeable material. The textile material increases the comfort of wearing the exoskin by the user. Due to the coating and/or lamination with the fluid-impermeable material, the displaceable fluid remains in the at least one fluid circuit of the exoskin.

In one aspect, the exoskin further comprises at least one of a pressure sensor, a trigger, and a power source. The exoskin further comprises at least one of an electronic sensor, a data collector, and a controller for controlling the at least one valve. These elements (electronic sensor, data collector and controller) support the proper utilization of the exoskin and avoid a misuse by sensing and controlling specific parameters as well as enhancing the basic function of the exoskin by providing additional features. For example, additional features are one of improved triggering of valves based on sensing beneath a sole of a user's foot for better timing and better control of valves, and collection of personal data like step count, movement statistics, gait parameters or fall detection.

In one aspect, the exoskin further comprises at least one actuator adapted to exert pressure on body parts of the user. With this feature, the exoskin is capable of giving haptic feedback to the user.

The exoskin as described herein can be used to support a movement of body parts of the user and to reduce fatigue of the user's muscles. Thus, the exoskin can be used for a wide range of purposes such as supporting people with limited mobility or people who do physically intensive work.

### DESCRIPTION OF THE FIGURES

A more complete appreciation of the invention and many of the attendant advantages thereof will be readily obtained as the same becomes better understood by reference to the following detailed description when considered in connection with the accompanying figures.

FIG. 1 illustrates a first aspect of a fluid circuit to be used for an exoskin.

FIG. 2 illustrates a schematic view of a regulation valve.

FIG. 3 illustrates a second aspect of the fluid circuit to be used for the exoskin.

FIG. 4 illustrates a schematic view of anchoring points positioned at a body part.

FIG. 5 illustrates a schematic view of an example of an exoskin comprising fluid circuits according to the second aspect.

FIG. 6 illustrates examples of a structure of the fluid circuit.

FIG. 7 illustrates examples of layers of the structure in FIG. 6.

FIG. 8 illustrates another example of the exoskin comprising the second aspect of the fluid circuit in connection with an elastic actuator.

FIG. 9 illustrates another example of the exoskin of FIG. 4.

FIG. 10 illustrates a detail view of the fluid circuit of FIG. 1 and FIG. 3 with a first arrangement of actuators.

FIG. 11 illustrates a detail view of the fluid circuit of FIG. 1 and FIG. 3 with a second arrangement of actuators.

FIG. 12 illustrates another example of the exoskin.

FIG. 13 illustrates an example of a compression garment for a lower limb including the exoskin.

FIG. 14 illustrates a first arrangement of fluid circuits in a compression garment of FIG. 13.

FIG. 15 illustrates an example of the structure of the fluid circuit in the compression garment of FIG. 13.

### DETAILED DESCRIPTION OF THE INVENTION

The invention will now be described based on the figures. It will be understood that the embodiments and aspects of the invention described herein are only examples and do not limit the protective scope of the claims in any way. The invention is defined by the claims and their equivalents. It will be understood that features of one aspect or embodiment of the invention can be combined with a feature of a different aspect or aspects and/or embodiments of the invention.

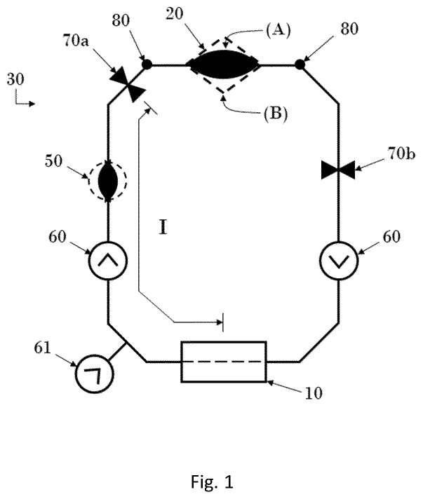

FIG. 1 illustrates a first aspect of a fluid circuit 30. The fluid circuit 30 is a circular fluid circuit 30 in which a direction of flow of a displaceable fluid 40 is unidirectional. The displaceable fluid 40 comprises, for example, water, an alcohol like propylene glycol, a polymer or oil, but this is not limiting of the invention. The direction of the flow of the displaceable fluid 40 can be set by a valve 60. The circular fluid circuit 30 comprises at least one valve 60 and in FIG. 1 two of the valves 60 are shown, but this is not limiting of the invention. In addition, the circular fluid circuit 30 can comprise also at least one regulation valve 70a or 70b. FIG. 1 shows two regulation valves 70a and 70b, but this is not limiting of the invention.

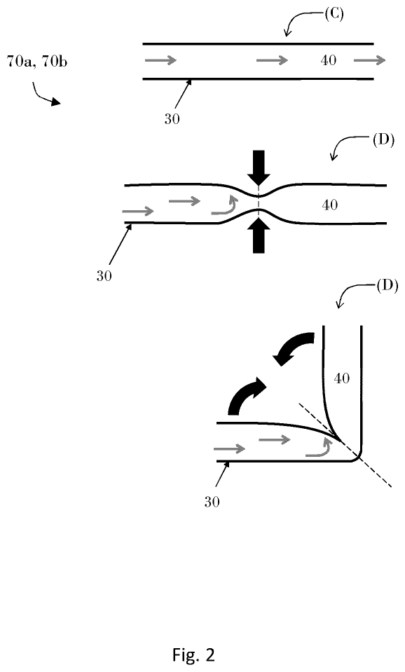

As seen in FIG. 2, the first regulation valve 70a and the second regulation valve 70b regulate the flow of the displaceable fluid 40 with an open state C or a closed state D. The regulation valve 70a, 70b is driven by a movement of the user and/or a position of the body of the user.

The regulation valve(s) 70a, 70b are bent, pinched, or obstructed by the movement of the user and thus the effect is to impede the flow of the displaceable fluid 40 in the fluid circuit 30 and thus the flow of the displaceable fluid 40 is regulated by the user.

The regulation valve(s) 70a and 70b comprise one of a pressure valve, angular valve or a NIMPLY gate. The pressure valve(s) 70a and 70b generate pressure, for example, by pinching a part of the fluid circuit 30 (shown by the arrows in the middle and bottom illustrations of FIG. 2). The angular valve blocks the flow of the displaceable fluid 40 partially or fully by throttling the fluid circuit 30 depending on a relative angle of two body parts 130 to which the fluid circuit 30 is affixed.

The NIMPLY gate (not illustrated) is a kind of pressure valve based on an additional actuator comprising two inputs and works as a logic control gate with an open state or a closed state. The NIMPLY gate comprises a fluid circuit side (first input) and a valve side (second input). If the additional actuator is filled with the displaceable fluid 40 via the valve side, the additional actuator of the NIMPLY gate obstructs the flow of the displaceable fluid through the fluid circuit 30. The NIMPLY gate is switched to the closed state and the flow of the displaceable fluid 40 within the fluid circuit 30 is cut-off. The NIMPLY gate is switched to the open state only if the displaceable fluid 40 is provided onto the fluid circuit side (first input) of the NIMPLY gate and not onto the valve side (second input). In this case the NIMPLY gate releasees the additional actuator which subsequently deflates. The obstruction to the flow of the displaceable fluid 40 through the fluid circuit side (first input) is thereby removed due to the deflation. Thus, the regulation valves 70a and 70b are actively switched between the open state or the closed state.

The circular fluid circuit 30 of FIG. 1 further comprises an interface 10, a fluid actuator 20 and a fluid bladder 50 which are fluidly connected to the circular fluid circuit 30.

If the user interacts with the interface 10, e.g., by compressing the interface 10, the displaceable fluid 40 in the interface 10 will be propelled outwards from the interface 10 towards the circular fluid circuit 30 in which, as a result, the fluid pressure is increased. It will be understood that the displaceable fluid 40 completely fills the whole circular fluid circuit 30.

Depending on the setting of the valves 60, the displaceable fluid 40 is forced to move, or is directed in a specific direction, towards the fluid bladder 50 and further in the direction of the first regulation valve 70a, the fluid actuator 20 and the second regulation valve 70b back to the interface 10. This is in a clockwise manner, as shown by the arrows of the valves 60. The displaceable fluid 40 could also move in an anti-clockwise manner in the circular fluid circuit 30.

If the next element in the circular fluid circuit 30 after the fluid bladder 50, namely the first regulation valve 70a is in a closed state, the fluid pressure is increased in a first section I between the interface 10 and the first regulation valve 70a. The fluid bladder 50 is made of an elastic material and can compensate for the increase of fluid pressure by expansion as indicated by the dotted lines. As a result, the fluid bladder 50 will be filled by the displaceable fluid 40 and thus is stretched or swells.

The filled fluid bladder 50 helps regulate the timing at which a force is applied to a body part 130. At the user interaction with the interface 10, the filled fluid bladder 50 can actuate the actuator 20 with a short delay in time when the valve 70a is switched to the opened state and thus delay the actuation.

In an aspect, the fluid bladder 50 comprises an internal one-way valve (not illustrated) which ensures that the flow of the displaceable fluid 40 is cut-off in the return direction back to the interface 10 when the fluid bladder 50 is stretched by the displaceable fluid 40. In a case, where the fluid bladder 50 does not include the internal one-way valve, the flow of the displaceable fluid 40 is cut-off by the valves 60. If the first regulation valve 70a switches from the closed state to the open state, the fluid bladder 50 deflates because of its elastic material pushing the displaceable fluid 40 towards the fluid actuator 20. The fluid actuator 20 is made of a flexible material able to stretch and swell and thereby change in volume. As a result, the fluid actuator 20 switches from a first state A to a second state B, wherein the second state B is different from the first state A and a second volume of the fluid actuator 20 is different in the second state B from a first volume in the first state A. The fluid actuator 20 is placed with at least one fixed anchoring point 80, at a body part 130 of the user, e.g., at the knee joint.

The fixed anchoring points 80 are points at which the fluid actuator 20 is affixed, fastened, tied, or connected in another way to one or more of the body parts 130 of the user. The fluid actuator 20 exerts the force at each of the fixed anchoring points 80, whereby the fixed anchoring points 80 transmit the force to the body part 130. Multiple ones of the anchoring points can coincide in one fixed anchoring point 80 or a movable anchoring point 81.

If the fluid actuator 20 switches from the first state A to the second state B, the force is applied—in this exemplary case—to the knee joint, and the extension movement of the knee is supported.

The at least one fixed anchoring point 80 could be at other parts of the user's body such as the hips, arms, neck, ankles, etc. Depending on the position of the at least one fixed anchoring point 80 at the body of a user, the same fluid actuator 20 can have a completely different effect and purpose. The fixed anchoring point 80 is a kind of fixation and not a fluidic element.

If the interface 10 is de-compressed by the user, the pressure in the fluid circuit 30 decreases allowing the displaceable fluid 40 to either return from the fluid actuator 20 back to the interface 10 (resulting in the fluid actuator 20 switching back to state A) or the regulation valves 70a and 70b are switched to the closed state keeping the fluid actuator 20 in the second state B until one of the first regulation valve 70a or the second regulation valve 70b is switched to the open state allowing the displaceable fluid 40 return back to the interface 10 and thus resulting in the fluid actuator 20 switching back to state A. If the displaceable fluid 40 returns to the interface 10, the system is reset, and the flow can start from the beginning.

The circular fluid circuit 30 can comprise a non-return valve 61. The non-return valve 61 can be placed at any position in the circular fluid circuit 30 to refill and/or replace the displaceable fluid 40 in the circular fluid circuit 30.

In a further aspect of the circular fluid circuit 30, it is also possible to provide the circular fluid circuit 30 without the fluid bladder 50. In this case, the regulation valve 70a would have to be in the open state while there is a user interaction with the interface 10. Consequently, no delay of actuation is possible and the regulation valve 70a would lose its purpose. To realise delay of actuation without the fluid bladder 50, the first valve 60 can be replaced with the regulation valve 70a. The regulation valve 70a is switched to the closed state when the user interaction with the interface 10 is over and to avoid a return flow of the displaceable fluid 40 back to the interface 10 until the regulation valves 70a, 70b or both are opened. The case where the regulation valve 70a is either again in the open state or remains in the open state, and the regulation valve 70b remains in the closed state, is equivalent to a second aspect of the fluid circuit 30.

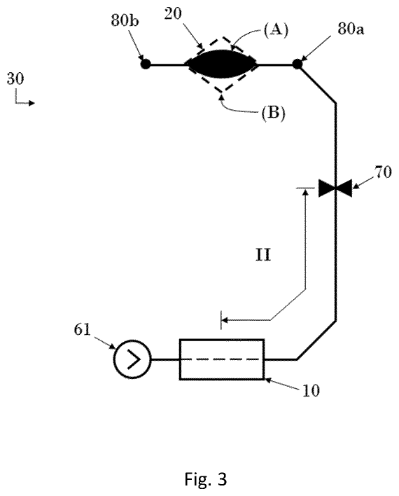

FIG. 3, illustrates a second aspect of the fluid circuit 30. The fluid circuit 30 of FIG. 3 is a linear fluid circuit 30. A direction of flow of the displaceable fluid 40 in the linear fluid circuit is two-stage bidirectional.

The two-stage bidirectional flow of the displaceable fluid 40 comprises a first stage, in which the flow of the displaceable fluid 40 is in a first direction. Upon occurrence of an event, the flow of the displaceable fluid 40 is reversed in a second stage and the displaceable fluid 40 changes the direction of flow in the linear fluid circuit 30.

For the sake of brevity, same elements will be referenced with same reference numbers in the figures and not described in detail again.

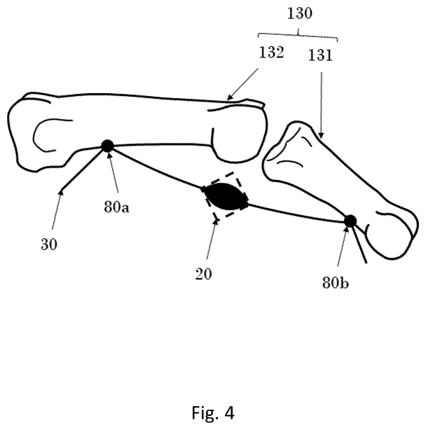

As seen in FIG. 3, the linear fluid circuit 30 comprises the interface 10, the regulation valve 70 and the fluid actuator 20 which is held in place by two fixed anchoring points 80a and 80b at two distinct locations of at least one of a body part 130 of the user to create the force between the two distinct locations. For example, as seen in FIG. 4 one of the locations could be a proximal phalange 131 of a finger of a body part 130 and the other one of the locations could be an intermediate phalange 132 of the finger of the body part 130. If the actuator 20 switches from one state A to B, or vice-versa, the force is applied, and the movement of the finger (body part 130) is supported. Other examples of the locations for the body part 130 are the ankle and leg or hand and forearm, but this is not limiting of the invention. To refill and/or replace the displaceable fluid 40 in the linear fluid circuit 30, the non-return valve 61 can be placed at any position in the linear fluid circuit 30.

If the user interacts with the interface 10, e.g., compresses the interface 10, the displaceable fluid 40 in the interface 10 will be propelled outwards of the interface 10 toward the linear fluid circuit 30 and thus starting the first stage of the process, and, as a result, the fluid pressure in the linear circuit 30 is increased. It will be understood that displaceable fluid 40 is the only fluid in the whole linear fluid circuit 30.

The displaceable fluid 40, as illustrated in FIG. 3, is forced to move, or is directed towards the fluid actuator 20, passing the regulation valve 70 if the regulation valve 70 is in an open state. In this second aspect, the fluid bladder 50 is an optional feature. The distance of an intermediate section II between the interface 10 and the regulation valve 70 could be sufficiently large that the increased fluid pressure at the interface 10 is absorbed in this intermediate section II. One example of a sufficiently large intermediate section II is the case, that the interface 10 is positioned at a heel of the foot of the user and the regulation valve 70 is positioned at an upper body part 130. For example, this upper body part 130 is of the shoulder and the fluid actuator 20 is positioned to support a movement of a shoulder.

As a result of the compression of the interface 10, the fluid actuator 20 switches from the first state A to the second state B. The force is applied to the body part 130 when the fluid actuator 20 switches from the first state A to the second state B. The force can be one of a positive force (push) or a negative force (pull).

As a first example of the event, which changes the first stage to the second stage of the two-stage bidirectional flow of the displaceable fluid 40, when the interface 10 is de-compressed by the user, the pressure in the linear fluid circuit 30 decreases causing the direction of flow of the displaceable fluid 40 to be reversed.

If the regulation valve 70 is in the open state, the displaceable fluid 40 returns from the fluid actuator 20 back to the interface 10 resulting in the fluid actuator 20 switching back to state A. If the regulation valve 70 is in the closed state, the fluid actuator 20 is kept in the second state B until the regulation valve 70 is switched to the open state allowing the displaceable fluid 40 to return to the interface 10 and switching the fluid actuator 20 back to state A. If the displaceable fluid 40 returns to the interface 10, the system is reset, and the flow can start from the beginning.

An exoskin 100 comprises at least one fluid circuit 30 according to the first aspect or the second aspect of the fluid circuit 30 described above. Depending on the application, the exoskin 100 can comprise at least one interface 10, at least one fluid actuator 20 and at least one of the valve 60 and the regulation valve 70, 70a or 70b. If desired, the at least one fluid circuit 30 can comprise none or at least one of fluid bladder 50 and/or non-return valve 61.

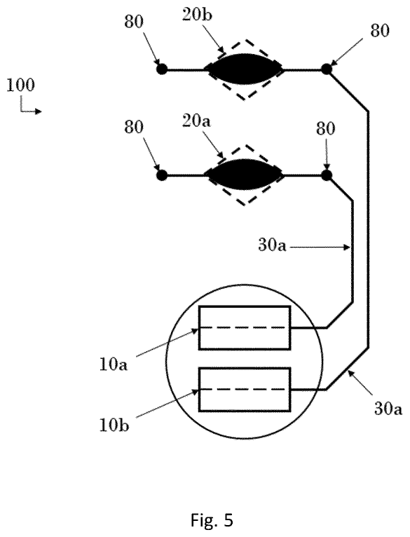

FIG. 5 illustrates a first example of the exoskin 100 comprising two linear fluid circuits 30 according to the second aspect. As illustrated, the at least two fluid circuits 30 are independent circuits and do not exchange the displaceable fluid 40 therebetween. The exoskin 100 comprises further two interfaces 10a and 10b and two actuators 20a and 20b with each having two fixed anchoring points 80.

If the interfaces 10 of the exoskin 100 of FIG. 5 are positioned, for example, with the first interface 10a at the heel of the user's foot and the second interface 10b at the ball of the user's foot, the exoskin 100 can perform two effects and/or purposes while the user is moving, e.g., walking or running. Thus, in a non-limiting example, a first one of the fluid circuits 30a of the exoskin 100 supports a contraction movement and the other one of the fluid circuits 30b of the exoskin 100 supports a bending movement. Depending on the position of the actuators 20 anchored with the fixed anchoring points 80 at the user's body, distinct parts of the user can be supported in their movement.

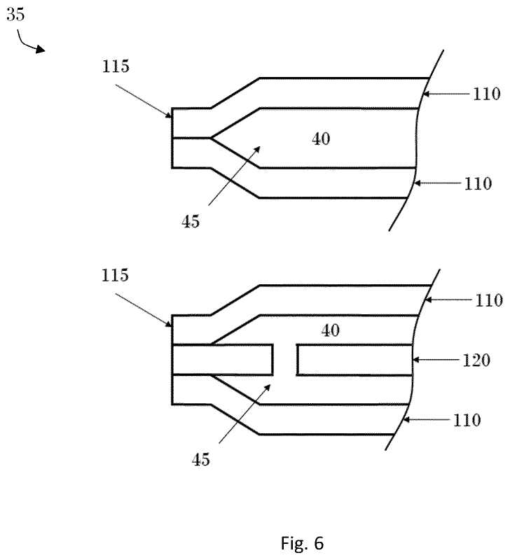

As seen in FIG. 6, a structure 35 of the fluid circuit 30 and/or the exoskin 100 is made of two external walls 110. The external walls 110 are affixed on their edges 115 to form an intermediate space 45. The intermediate space 45 accommodates the displaceable fluid 40. In a case, the structure 35 comprises intermediate layers 120 which are affixed to both of the edges 115 of the external walls 110. In another case (not illustrated), the intermediate layers 120 are affixed to only one of the external walls 110. However, in both cases the presence of the intermediate layers 120 may vary throughout the fluid circuit 30 and/or the exoskin 100. The intermediate layers 120 comprise holes which steer and/or direct the flow of the displaceable fluid 40 within the intermediate space 45 of the fluid circuit 30.

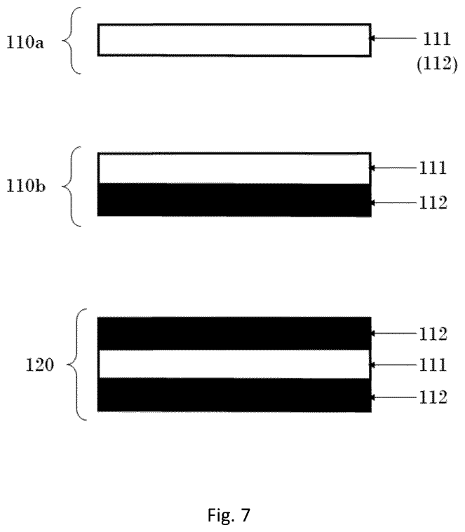

The external walls 110 and the intermediate layers 120 of the structure 35 of the fluid circuit 30 and/or the exoskin 100 are made of at least one layer as shown in FIG. 7.

In one example, an external wall 110a is made of a single polymer layer 112 in a case in which the external wall 110a is not an outermost layer of the fluid circuit 30. In a further example, the external wall 110a is a single textile layer 111 in a case in which the external wall 110a is the outermost layer of the fluid circuit 30 and/or the exoskin 100. The polymer layer 112 can be made of a thermoplastic elastomer, such as Thermoplastic Polyurethane (TPU) and/or Styrene-Ethylene-Butylene-Styrene (SEBS), but this is not limiting of the invention. The textile layer 111 can be made of one of a woven, non-woven, knitted, elastic or inelastic material, such as cotton, wool, linen, nylon, viscose, rayon, neoprene, polyamide, polyester, elastane or expanded polytetrafluoroethylene, but this is not limiting of the invention. In yet a further example, the external wall 110b is made of a multi-layer material of at least one of a polymer layer 112 and a textile layer 111. In this example, an innermost layer is the polymer layer 112 and the outermost layer is the textile layer 111. It will be understood that the structure 35 can vary locally based on the application of the exoskin 100.

The structure 35 having the multi-layer material comprises at least one of an elastic polymer layer, an elastic support layer, a polymer layer with low elasticity or a textile layer with low elasticity. Thus, the elasticity, comfort, and other properties of the exoskin 100 can be set and varied locally to improve adaptability to the anatomy of the body part 130 of the user.

A local elastic behaviour of the exoskin 100 can be influenced by cutting windows or holes in the low elasticity layers. In addition, at least one of the layers can comprise one of perforations or patterning on a local scale, which give additional properties to the exoskin 100 such as increased elasticity, visual effects, or improved force reaction between the exoskin 100 and the body part 130 of the user, but this is not limiting the invention.

In an aspect, the structure 35 comprises supports (not illustrated). The supports can be rigid or flexible and are placed within the structure 35 or onto the structure 35. The supports partially limit a transverse swelling and can be placed at least to one of the fluid bladder 50, the interface 10 and the fluid actuator 20 for setting a threshold for the swelling.

The textile material raises the comfort of wearing the exoskin and ensures at the same time due to a coating and/or lamination with a fluid-impermeable material, that the displaceable fluid 40 remains in the at least one fluid circuit 30 of the exoskin 100.

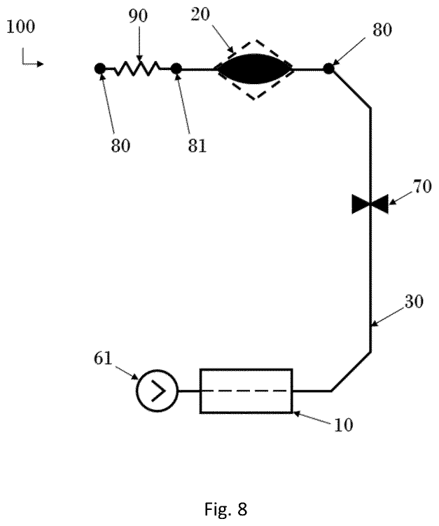

In FIG. 8 another example is shown of the exoskin 100 comprising the second aspect of the linear fluid circuit 30 in connection with an elastic actuator 90. The exoskin 100 of FIG. 8 is a further development of the second aspect shown in FIG. 3.

The fluid actuator 20 is anchored on one side at the fixed anchoring point 80. On the other side, the fluid actuator 20 is anchored with at least one movable anchoring point 81. The elastic actuator 90 is connected on one side to the movable anchoring point 81 and on the other side to the fixed anchoring point 80. The movable anchoring point 81 is a so-called force modifier and connects the elastic actuator 90 to the fluid actuator 20. The movable anchoring point 81 influences the length of the elastic actuator 90 and thus the magnitude of the force applied on the fixed anchoring point 80 of the elastic actuator 90 or the angle at which forces are applied on the fixed anchoring point 80 of the elastic actuator 90. The elastic actuator 90 is a non-fluidic actuator and not in fluid connection to the fluid circuit 30.

With this design of FIG. 8, the change of the state A to the state B of the fluid actuator 20, and vice-versa, results in an application of the force onto the movable anchoring point 81 causing a linear movement and thus an absorption of energy of the elastic actuator 90. When the flow of the displaceable fluid 40 is reversed by the event, the energy in the elastic actuator 90 is released.

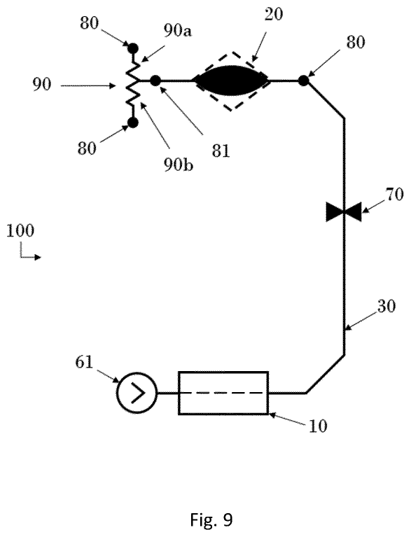

As seen in FIG. 9, another example of the exoskin 100 of FIG. 8 is shown. In this example, the movable anchoring point 81 connects the fluid actuator 20 to an intermediate position of the elastic actuator 90 and divides the elastic actuator 90 into a first elastic actuator 90a and a second elastic actuator 90b. The first elastic actuator 90a and the second elastic actuator 90b can have different lengths. The change of state of the fluid actuator 20 causes the movable anchoring point 81 to bend the elastic actuator 90 which results in the possibility to influence the angle and magnitude (positive and negative) of the forces applied on the fixed anchoring points 80. In another example, one or more movable anchoring points 81 can be connected to two or more elastic actuators 90. With the connection of elastic actuators 90, 90a, 90b to the fluid circuit 30, the exoskin 100 can be configured to support the movement of body parts 130 of users in an individual way.

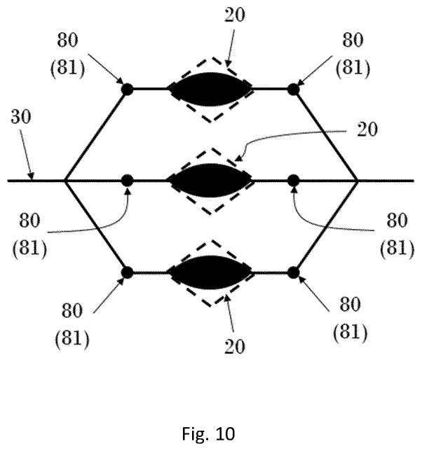

To further individualize the exoskin 100 to the needs of the user, a plurality of actuators 20 can be arranged and connected in a desired way in/to the fluid circuit 30 of the exoskin 100. As seen in FIG. 10, the plurality of actuators 20 of the fluid circuit 30 can be arranged in a parallel arrangement. The exoskin 100, comprising at least one fluid circuit 30 with the parallel arrangement of actuators 20, can maximize the applied force to the body part 130 of the user, when the plurality of parallel actuators 20 switches from state A to state B, and vice-versa.

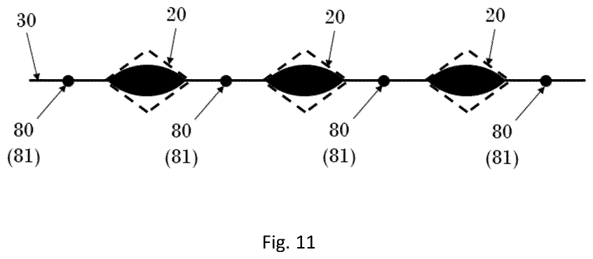

In another example for further individualization of the exoskin 100 to the needs of the user, the plurality of actuators 20 can be also arranged and connected in/to the fluid circuit 30 in a serial arrangement, as shown in FIG. 11. The exoskin 100, comprising at least one fluid circuit 30 with the serial arrangement of actuators 20, can maximize the distance of movement of the body part 130 of the user, when the plurality of serial actuators 20 switches from state A to state B, and vice-versa.

In another example for further individualization of the exoskin 100 to the needs of the user (not illustrated), a plurality of fluid bladders 50 can be also arranged and connected in/to the fluid circuit 30 in a serial or parallel arrangement. Thus, an optimal ratio between a surface to a volume ratio of the fluid bladders 50 could be achieved. A plurality of interfaces can be also arranged and connected in the fluid circuit 30 in a serial or parallel arrangement. Thus, a ratio between a surface to a volume of the interface 10 could be further optimized which results in finer or broader placement and energy harvesting, as well as improves the timing and/or interaction to actuation ratio.

In the parallel arrangement as well as the serial arrangement of the plurality of actuators 20, the fixed anchoring points 80 and the movable anchoring points 81 need to be placed such at the body part 130 of a user that the plurality of actuators 20 apply the force to the body part 130 of the user. It will be understood that the placement of the anchoring points 80 and 81 is based on a logic approach and thus will be not described in detail here.

It will be apparent, that the parallel arrangement and the serial arrangement of the plurality of actuators 20 can be combined in a modified example, to further individualize the support of the movement of a user by the exoskin 100.

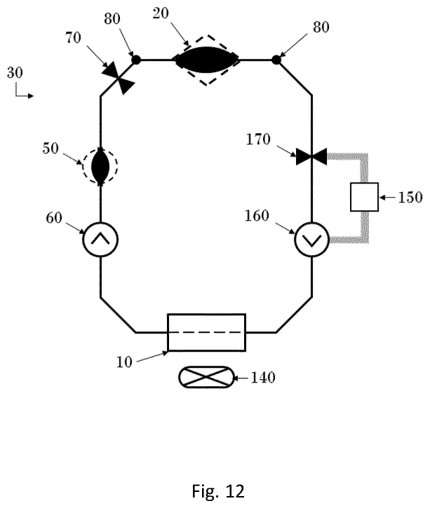

FIG. 12 illustrates a modified example of the exoskin 100 comprising embedded electronics such as an electronic sensor 140 and a controller 150 for controlling at least one of electronic valve(s) 160 and 170. The electronic sensor 140, controller 150 and the electronic valves 160 and 170 support a proper utilization of the exoskin 100 and avoid misuse by sensing and controlling specific parameters as well as enhancing the basic function of the exoskin 100 by providing additional features. Additional features are, for example, an improved triggering of electronic valves 160, 170 based on pressure sensing with the electronic sensor 140 beneath a sole of a user's foot for better timing and better control of the electronic valves 160, 170, and collection of personal data like step count, movement statistics, muscular activity, gait parameters or fall detection, but this is not limiting of the invention. The electronic sensor 140 can be one of a sensor for muscular activity, temperature or moisture, or a combination thereof. The controller 150 collects data generated by the electronic sensor 140 and controls the electronic valve(s) 160, 170 with a pre-defined delay. The electronic sensor 140 and the controller 150 are connected wirelessly or with a cable. Energy is supplied by a power source (not illustrated). It will be understood that the modified example of the exoskin 100 may comprise one or more electronic sensors 140, one or more controllers 150 and one or more electronic valve(s) 160, 170.

The exoskin 100 according to the present disclosure can be used to individually support the movement of body parts 130 of a user and/or reduce the fatigue of muscles. Consequently, the exoskin 100 can be applied for a wide range of purposes such as supporting users with limited mobility or for rehabilitation purposes, or people who do physically intensive work. Depending on the field of application and the desired size of the exoskin 100, the exoskin 100 can be also understood as exosuit, exoskeleton, interface device or wearable device. Further, the exoskin 100 is not limited to be used by humans but could also be applied to animals for veterinary medicine purposes.

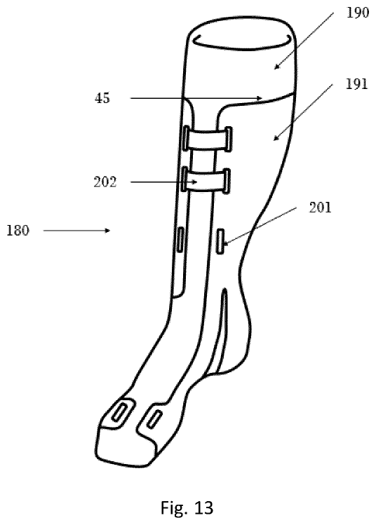

The exoskin 100 can be integrated into a compression garment 180. For example, FIG. 13 shows the compression garment 180 placed on a lower limb, such as a leg or a foot, but it will be appreciated that the compression garment 180 can also be placed on other parts of the body, for example on an arm. The compression garment 180 can be, for example, a knitted sock, kinesiology tape, or a bandage.

In one aspect, the compression garment 180 is placed on a part of the body, wrapped around a part of a body, or attached directly to a part of the body.

The compression garment 180 comprises a bottom layer 190 and a top layer 191. One or more cavities 45 are formed between the bottom layer 190 and the top layer 191. One or more fluid circuits 30 can be placed in the one or more cavities 45. In FIG. 13 a single cavity 45 is shown, but this is not limiting of the invention.

In one aspect, the top layer 191 and the bottom layer 190 are formed by a single layer of the compression garment 180. This single layer can be made of a textile. In another aspect, the top layer 191 and the bottom layer 190 are formed of two distinct layers. The top layer 191 can comprise for example a plain textile, a coated textile, a laminated textile or similar. The textile can comprise natural fibers, for example cotton or wool, or synthetic fibers, like polyamide, polyester, viscose, or rayon. The textile may also comprise a mixture of natural fibers and synthetic fibers. The compression garment 180 can be made from knitted, woven, or non-woven fibers. The top layer 191 can be attached to the bottom layer 190 for example through sewing, welding, gluing, or through hoop or loop tape for example.

In one aspect, the top layer 191 and the external wall 110 overlap and/or the bottom layer 190 and the external wall 110 overlap.

In one aspect, the exoskin 100 and the compression garment 180 are combined to form a single item.

In one aspect, the exoskin 100 has fastening straps 202. The fastening straps 202 can be attached to the top layer 191, the bottom layer 190 or to the external wall 110. The fastening straps 202 are used to improve a fit of the compression garment 180 and the exoskin 180 as well as ensuring the correct placement of the circuit 30 on the body. In one aspect, the bottom layer 190 and/or the top layer 191 have openings 201 to allow the fluid circuit 30 to be introduced and/or extracted from the exoskin 100. The fluid circuit 30 can also be bonded to the exoskin 100, for example by sewing, welding, gluing.

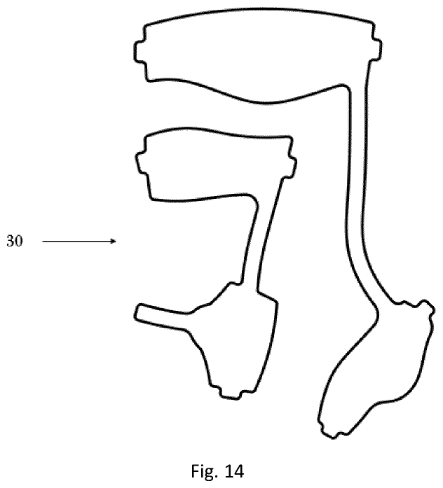

FIG. 14 illustrates one arrangement of the compression garment 180 with two of the fluid circuits 30 but this is not limiting of the invention and only one single fluid circuit 30 can be used.

The bottom layer 190 and the top layer 191 form a first intermediate space 45.

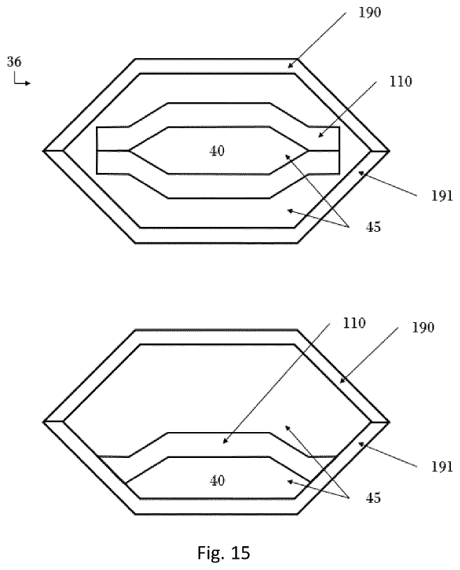

In a first aspect shown in the top half of FIG. 15, two external walls 110 as seen in FIG. 6 are located in the first intermediate space 45 forming a second intermediate space 45 throughout which the displaceable fluid 40 flows.

In a second aspect shown in the bottom half of FIG. 15, an external wall 110 is directly bonded to the bottom layer 190 (not shown) or to the top layer 191 by, for example sewing, welding, or gluing, forming a second intermediate space 45 through which the displaceable fluid 40 flows.

The compression garment 180 can be used as an active massage device and/or for absorbing shocks.

### REFERENCE NUMBERS

100 exoskin
10 interface
20 fluid actuator
30 fluid circuit
35 structure
36 structure of the exoskin
40 displaceable fluid
45 intermediate space
50 fluid bladder
60 valve
61 non-return valve
70 regulation valve
80 fixed anchoring point
81 movable anchoring point
90 elastic actuator
110 external wall
111 textile layer
112 polymer layer
115 edges
120 intermediate layer
130 body part
140 electronic sensor
150 controller
160 electronic valve
170 electronic valve
180 compression garment
190 bottom layer
191 top layer
201 openings
202 fastening straps

1. An exoskin for a user, the exoskin comprising:
at least one fluid circuit, comprising a displaceable fluid;
at least one interface fluidly connected to the fluid circuit;
at least one fluid actuator fluidly connected to the fluid circuit; and wherein the at least one fluid actuator is changeable in volume switches from a first state with a first volume to a second state with a second volume, different from the first state, in reaction of a flow of the displaceable fluid caused by an interaction of the user with the at least one interface.

2. The exoskin of claim 1, wherein the at least one fluid actuator is made of a flexible material.

3. The exoskin of claim 1, further comprising a valve located in the at least one fluid circuit for forcing the displaceable fluid in a specific direction.

4. The exoskin of claim 1, further comprising a fluid bladder located in the at least one fluid circuit for compensating an increase of fluid pressure in the circuit.

5. The exoskin of claim 1, further comprising at least one fixed anchoring point for connecting the at least one actuator to one or more body parts of the user for applying a force.

6. The exoskin of claim 5, further comprising at least one elastic actuator and at least one movable anchoring point, wherein the at least one elastic actuator is connected to the at least one actuator via the at least one movable anchoring point.

7. The exoskin of claim 5, wherein the at least one elastic actuator is connected to the at least one fixed anchoring point, wherein the at least one elastic actuator is adapted to change a magnitude and angle of the force applied on the at least one fixed anchoring point.

8. The exoskin of claim 1, wherein the at least one fluid circuit comprises a structure, wherein the structure comprises two external walls and the two external walls are affixed on their edges to form an intermediate space for accommodation of the displaceable fluid.

9. The exoskin of claim 1, further comprising a regulation valve, wherein the regulation valve is adapted to be bent, pinched, or obstructed by a movement of the user and thereby to impede the flow of the displaceable fluid in the fluid circuit.

10. The exoskin of claim 8, wherein the external walls comprise at least one layer made of at least one of a polymer material or a textile material.

11. The exoskin of claim 1, further comprising at least one of an electronic sensor, a controller and an electronic valve.

12. The exoskin of claim 1, wherein the displaceable fluid is one of an aqueous solution, an alcohol, oil, or a polymer.

13. The exoskin of claim 1, wherein the at least one fluid circuit is one of a circular fluid circuit or a linear fluid circuit.

14. (canceled)

15. A compression garment integrating an exoskin for a user and comprising a bottom layer and a top layer, the exoskin comprising:
at least one fluid circuit, comprising a displaceable fluid;
at least one interface fluidly connected to the fluid circuit
at least one fluid actuator fluidly connected to the fluid circuit and
wherein the at least one fluid actuator is changeable in volume switches from a first state with a first volume to a second state with a second volume, different from the first state, in reaction of a flow of the displaceable fluid caused by an interaction of the user with the at least one interface.

16. The compression garment of claim 15, wherein the exoskin comprises one or more openings to enable the fluid circuit to be introduced and extracted from the exoskin.

17. The compression garment of claim 15, wherein the compression garment is adapted to be placed on a lower limb.
